(19) United States
(12) Patent Application Publication
(10) Pub. No.: US 2025/0135375 A1
(43) Pub. Date: May 1, 2025

(54) METHODS FOR REACTIVE CRYSTALLISATION

(71) Applicant: Micropore Technologies Limited, Redcar (GB)

(72) Inventors: Matt Bennett, Redcar (GB); Elina Beveniou, Redcar (GB); Alex Kerr, Redcar (GB); David Hayward, Redcar (GB)

(73) Assignee: Micropore Technologies Limited, Redcar (GB)

(21) Appl. No.: 18/833,499

(22) PCT Filed: Jan. 25, 2023
(86) PCT No.: PCT/GB2023/050157

§ 371 Date: Jul. 26, 2024

(30) Foreign Application Priority Data
Jan. 26, 2022 (GB) 2201007.8

(51) Int. Cl. B01D 9/00; A61K 9/16; B01D 63/06; B01D 69/02; C01B 25/32; C01F 11/18; C01F 11/46

(52) U.S. Cl. CPC B01D 9/005; A61K 9/1611; A61K 9/1682; B01D 63/06; B01D 69/02; C01B 25/327; C01F 11/185; C01F 11/468; B01D 2009/0086; B01D 2315/10; B01D 2325/02

(57) ABSTRACT

There is described a method of preparing solid particles of a material, said method comprising controlling provision of a first liquid phase, wherein said first liquid phase comprises a solution of a first material through a membrane, said membrane defining a plurality of pores; and controlling provision of a second liquid phase, wherein said second liquid phase comprises a solution of a second material; reacting the first a second materials to produce a third liquid phase comprising a solution of a third material; and supersaturating the third liquid phase to form solid particles of the third material.

### FIELD OF THE INVENTION

The present invention relates to a novel method of preparing particulate materials using a mixing reactor and novel apparatus towards for example, in reactive crystallisation.

More particularly, the present invention relates to a mixing reactor and a method of using such a reactor, for example, in producing particles such as micro/nanoparticles or metal-organic frameworks, or inorganic materials.

### BACKGROUND TO THE INVENTION

Reactive crystallisation is caused when a chemical reaction results in the supersaturation of a crystallising compound.

These types of crystallisation reactions cause high local supersaturations which results in high nucleation rates and small sized crystals in the submicron to several micron range.

The chemical manufacturing process industry is highly invested in the production of crystals of small sizes to improve properties such as chemical reactivity, dissolution rate, bioavailability, and to avoid additional downstream operations such as milling to reduce the particle size.

Reactive crystallisation combines both synthesis of a material as well as a controlled crystallisation step to influence the crystal sizes, Crystal Size Distribution (CSD) and material purity.

In the chemical production industries, there is considerable interest in combining chemical reactions and controlled crystallisation in a single hybrid process.

Traditional chemical manufacturing plants have the chemical reaction process followed by recrystallisation separately. However, in the case of reactive crystallisation the final chemical reaction towards a desired product, and its controlled crystallisation towards desired sizes and morphologies, is occurring simultaneously.

Chemical reaction processes in industry tend to involve multi-step reactions where reagents are mixed together towards obtaining desired products. These reactions tend need to be carried out under specific conditions such as temperature, pH range, pressures and so forth. After the reaction step further process are then carried out for the separation and purification of the production through either side-products or residual solvents. One such type of process for solid material is crystallisation.

Crystallisation process design in industry aims at controlling the size, shape, purity and polymorphic form of crystals. In order to produce crystals of small mean size directly, isolation techniques and equipment configurations producing uniformly high supersaturation, i.e. high rates of nucleation are needed.

By having both processes occur at the same time this avoids the need for separate reaction and recrystallisation steps which not only increase time and cost along with the need for additional reagents and energy.

Production of crystals of small size in the manufacturing process of compounds is important for improving and obtaining desired properties. An example for pharmaceutical compounds is to improve properties such as dissolution rate, bioavailability, and tableting of the drugs, and to avoid additional downstream operations such as milling to reduce the particle size. Furthermore, many new pharmaceuticals have low water solubility. It is estimated that around half of the new active pharmaceutical ingredients (APIs) being identified are either insoluble or poorly soluble in water. Therefore, solving bioavailability problems is a major challenge for the pharmaceutical industry.

Studies with poorly water-soluble drugs demonstrate that particle size reduction to the sub-micron range can lead to an increase in the dissolution rate and a higher bioavailability. Furthermore, reactive crystallisation can lead to the formation of an exclusive metastable polymorph of a pharmaceutical API.

Metal, metal oxides and metal salt particles with micro to nanometre scale dimensions have a wide range of applications, including (but not limited to) catalysts, pigments, polishes, ultraviolet absorbers and in ceramics.

Such particles can be formed by chemical reaction of aqueous solutions of metal salts under conditions such as heated, pressurised, or supercritical water.

This methodology for the reaction of metal salts in aqueous solutions offers distinct advantages over other methods for micro/nanoparticle creation such as (but not limited to) jet milling in terms of cost and viability. This is due to this reaction methodology and crystallisation being able to be performed as a continuous process.

However, it is difficult to perform this type of reaction process on a commercial scale utilising current methods due to existing reactor configurations being unable to control precipitation reactions effectively leading to frequent blockage of the reactor and inadequate control of particle size, shape as well as product purity.

Therefore, the design of a reactor where, for example, a water and salt solution mix can be of crucial importance to the size and properties of the nanoparticles produced.

Inorganic materials, which are generally considered to be those, for example, that are not based on carbon, can also be crystallised using as reactive crystallisation techniques.

European Patent application No. 3 071 320 (University of Nottingham/Promethean Particles) describes a counter-current mixing reactor for the formation of solid nanoparticles. The reactor comprises a body provided with an inner passage, a first inlet, a second inlet and an outlet, the inner passage is connected to the first inlet. The body also has an outer passage running from the second inlet wherein the outer passage enters the inner passage at an angle of 90 degrees such that, in use, the mixing reactor introduces the second fluid perpendicular to the flow of the first fluid. The reactor utilises turbulent mixing and does not make use of a membrane.

### SUMMARY OF THE INVENTION

There is a need for an improved method of reactive crystallisation, i.e. chemical reaction with controlled crystallisation, and improved apparatus for reaction crystallisation. The method should be capable of being scaled up if desirable and can optionally be continuous process.

Thus, the present invention allows the scale up and/or continuous production of small and non-aggregated solid particles by conventional reactive techniques, e.g. precipitation of salts towards desired materials. It will be understood by the person skilled in the art the reagents and solvents to use towards the chemical reaction that will induce supersaturation towards controlled crystallisation of materials.

We have surprisingly found that membrane emulsification apparatus can be utilised to generate laminar mixing of liquid reactants thus creating a reactive mixing environment.

Furthermore, it has been surprisingly found that a crossflow membrane emulsification apparatus (AXF), utilising a tubular membrane, can suitably be used for the reactive production of solid particles.

According to a first aspect of the invention there is provided a method of preparing solid particles of a material, said method comprising controlling provision of a first liquid phase, wherein said first liquid phase comprises a solution of a first material through a membrane, said membrane defining a plurality of pores; and controlling provision of a second liquid phase, wherein said second liquid phase comprises a solution of a second material; reacting the first a second materials to produce a third liquid phase comprising a solution of a third material; and supersaturating the third liquid phase to form solid particles of the third material.

It will be understood that the reaction between the first and second materials takes place when the solution of the first material passes through the membrane and comes in contact with the solution of the second material.

According to a further aspect of the invention there is provided a method of preparing solid particles of a material, said method comprising controlling provision of a first liquid phase, wherein said first liquid phase comprises a solution of a first material, in a first flow direction to a membrane, said membrane defining a plurality of pores; and controlling provision of a second liquid phase, wherein said second liquid phase comprises a solution of a second material, introduction of the second liquid phase being substantially perpendicular to the introduction of the first liquid phase; reacting the first a second materials to produce a third liquid phase comprising a solution of a third material; and supersaturating the third liquid phase to form solid particles of the third material.

The methods of the invention may comprise the reactive preparation of solid particles of a material in crystalline form or amorphous form, or a combination thereof.

In one aspect of the invention the method comprises the reactive preparation of solid crystalline particles of a material. In another aspect of the invention the method comprises the reactive preparation of solid amorphous particles of a material.

According to a further aspect of the invention there is provided a method of reactive preparation of solid particles of a third material, said method comprising reacting a first liquid phase with a second liquid phase by dispersing the first liquid phase in the second liquid phase;

wherein said first liquid phase comprises a solution of a first material and said second liquid phase comprises a solution of a second material;

said method comprising controlling provision of the first liquid phase in a first flow direction to a membrane, said membrane defining a plurality of pores; and

controlling provision of the second liquid phase to the membrane in a crossflow (AXF) to the first flow direction, via the plurality of pores, to reactively form a solution of a third material; and optionally supersaturating the solution of the third material in order to produce particles of the third material.

It will be understood by the person skilled in the art that the term “crossflow” as used herein shall mean substantially perpendicular, i.e. at an angle of 90 degrees to the first flow direction, plus or minus 45 degrees. Thus according to this aspect of the invention there is provided a method as herein described wherein the “crossflow” of the second liquid phase is at an angle of 90 degrees to the flow direction of the first liquid phase, plus or minus 45 degrees.

According to a further aspect of the invention there is provided a method of reactive preparation of solid particles of a material, said method comprising dispersing a first liquid phase, comprising a solution of a first material, in a second liquid phase, comprising a solution of a second material;
wherein said method uses a crossflow reactive mixing apparatus; said crossflow reactive mixing apparatus comprising:
an outer tubular sleeve provided with a first inlet at a first end; a material outlet; and a second inlet, distal from and inclined relative to the first inlet;
a tubular membrane provided with a plurality of pores and adapted to be positioned inside the tubular sleeve; and
optionally an insert adapted to be located inside the tubular membrane, said insert comprising an inlet end and an outlet end, each of the inlet end and an outlet end being provided with chamfered region; the chamfered region is provided with a plurality of orifices and a furcation plate; and
controlling provision of the first liquid phase to the tubular membrane; and controlling provision of a second liquid phase to the tubular membrane via the plurality of pores to reactively form a solution of a third material.

It will be understood that the method of the invention may comprise preparing solid particles of more than one material, e.g. as co-crystals, comprising two or more components, and which may form a unique crystalline structure with unique properties.

The prepared reactive solution (i.e. after reaction) will include one or more dissolved materials. A variety of dissolved materials may be subjected to the method of the present invention. Typically, the dissolved material may be one or more organic compounds, which may include, for example, unreactive reagents, pharmaceutically active compounds, bioactive agents, nutraceuticals, polymers and the like; metal-organic frameworks; or inorganic materials.

According to one aspect of the invention the prepared reacted solution (i.e. after reaction) will include a dissolved material, wherein the material is an organic compound.

In another aspect of the invention the prepared reacted solution (i.e. after reaction) will include a dissolved material, wherein the material is an inorganic material.

This method of the invention may comprise the reactive preparation of solid particles of a material in crystalline form or amorphous form, or a combination thereof. In one aspect of the invention the method comprises the reactive preparation of solid crystalline particles of a material. In another aspect of the invention the method comprises the reactive preparation of solid amorphous particles of a material.

In a particular aspect of the invention dissolved prepared material (i.e. after reaction) comprises a material of low solubility. The term “low solubility” should be construed as meaning materials of poor bioavailability due to low water solubility. Up to 90% of the active pharmaceutical substances under development are poorly water soluble, usually resulting in low bioavailability.

In one aspect of the invention the term “soluble” shall mean from 10 to 30 parts solvent is needed to dissolve 1 part solute. The term “low solubility” shall mean from 100 to 10, 000 parts solvent is needed to dissolve 1 part solute. The term “slightly soluble” shall mean from 100 to 1,000 parts solvent is needed to dissolve 1 part solute. The term “insoluble” shall mean more than 10,000 parts solvent is needed to dissolve 1 part solute. These terms are generally defined by the US Pharmacopeia.

Reactive crystallisation uses miscible solvents where the reagents and by-products are all soluble in. The term “solvent” is used herein to describe a solvent or a mixture of solvents wherein the material of interest is at least slightly soluble, as defined by US Pharmacopeia. The desired product will, however, be insoluble as it is formed and crystallised out. This also provides a means of assisting in the separation process. It will be understood by the person skilled in the art the type of solvents to use for the reactive crystallisation of material.

The ratios and amounts of those materials may be adjusted according to the material, solvents and physicochemical properties, such as solubility, melting point, etc.

Reactive crystallisation can be affected by the addition of surfactants, which may play a role in nucleation and growth kinetics and may modify size distribution of crystalline and amorphous particles. In addition, the addition of surfactants may modify the crystal polymorph and particle morphology. The solution solvent may additionally comprise one or more surfactants or co-surfactants.

The surfactants may be selected from one or more of non-ionic surfactants, anionic surfactants, cationic surfactants and zwitterionic surfactants; and combinations thereof.

Non-ionic surfactants used in the present invention may be selected from, but shall not be limited to, polyvinyl alcohol (PVA); hydroxy propyl methyl cellulose (HPMC); poly(ethylene glycol)-block-poly(propylene glycol)-block-poly(ethylene glycol); Pluronic P123 (PEO-PPO-PEO); ethoxylates, including fatty alcohol ethoxylates, such as, octaethylene glycol monodecyl ether, pentaethylene glycol monodecyl ether and hexoxy ethylene glycol mono-n-dodecyl ether; alkylphenol ethoxylates, such as, Triton X-100; fatty acid esters, such as, glycerol monostearate and glycerol monolaurate; fatty acid esters of sorbitol, such as, sorbitan monolaurate, sorbitan monostearate and sorbitan tristearate; fatty acid amides, such as, cocamide monoethanolamine and cocamide diethanolamine; and Tween, e.g. Tween 20, a non-ionic detergent widely used in biochemical applications, Tween 40, Tween 60 and Tween 80; and ethoxylates, including fatty alcohol ethoxylates, such as, octaethylene glycol.

Anionic surfactants may be selected from, but shall not be limited to, sodium dodecyl sulfate (SDS), sodium dodecyl benzene sulfonate (SDBS), ammonium lauryl sulfate, and sodium bis(2-ethyl hexyl) sulfosuccinate.

Cationic surfactants may be selected from, but shall not be limited to, ammonium salts, such as, cetyl trimethyl ammonium bromide (CTAB), cetylpyridinium chloride (CPC), benzalkonium chloride (BAC), benzethonium chloride (BZT), dimethyl dioctadecyl ammonium chloride, dioctadecyl dimethyl ammonium bromide (DODAB) and dodecyl dimethyl ammonium bromide (DDAB).

Zwitterionic surfactants may be selected from, but shall not be limited to phospholipids, such as, phosphatidyl serine, phosphatidyl choline (PC) and phosphatidyl ethanolamine (PE).

The amount of surfactants that is required for achieving good particle size and shape may vary, but may be from about 0.005% to 2.0% w/w of the total solution.

Surfactants and co-surfactants include, but shall not be limited to, for example, Tween, a non-ionic detergent widely used in biochemical applications. It is also known as PEG (20) Sorbitan monolaurate. Other emulsifiers include poloxomer, a hydrophilic non-ionic surfactant which is a non-ionic triblock copolymer, Tween 80 and lecithins.

In the method of the present invention the crossflow membrane reactive crystallisation uses the flow of a second phase, to detach droplets from the membrane to sweep and evenly mix flows of a first phase coming through the membrane pores. This contrasts with the use of turbulent flow, e.g. by stirring, for solid particle production.

The position of the particle outlet may vary depending upon the direction of flow of the first liquid phase, i.e. from inside the membrane to outside or from outside the membrane to inside. If the flow of the first liquid phase is from outside the membrane to inside then the particle outlet will generally be at a second end of the tubular sleeve. If the flow of the first liquid phase is from inside the membrane to outside then the particle outlet may be a side branch or at the end.

In one aspect of the invention the crossflow apparatus includes an insert as herein described and the first inlet is a first liquid phase first inlet and the second inlet is a second liquid phase inlet; such that the first liquid phase travels from outside the tubular membrane to inside.

In another aspect of the invention the crossflow apparatus does not include an insert and the first inlet is a first liquid phase inlet and the second inlet is a second liquid phase inlet; such that the first liquid phase travels from inside the tubular membrane to outside.

When an insert is present and the tubular membrane is positioned inside the outer sleeve, the spacing between the insert and the tubular membrane may be varied, depending upon the laminar conditions desired, etc. Generally, the insert will be located centrally within the tubular membrane, such that the spacing between the insert and the membrane will comprise an annulus, of equal or substantially equal dimensions at any point around the insert. Thus, for example, the spacing may be from about 0.05 to about 10 mm (distance between the outer wall of the insert and the inner wall of the membrane), from about 0.1 to about 10 mm, from about 0.25 to about 10 mm, or from about 0.5 to about 8 mm, or from about 0.5 to about 6 mm, or from about 0.5 to about 5 mm, or from about 0.5 to about 4 mm, or from about 0.5 to about 3 mm, or from about 0.5 to about 2 mm, or from about 0.5 to about 1 mm.

When the tubular membrane is positioned inside the outer sleeve, the spacing between the tubular membrane and the outer sleeve may be varied, depending upon the size of droplets desired, etc. Generally, the tubular membrane will be located centrally within the outer sleeve, such that the spacing between the membrane and the sleeve will comprise an annulus, of equal or substantially equal dimensions at any point around the tubular membrane. Thus, for example, the spacing may be from about 0.5 to about 10 mm (distance between the outer wall of the membrane and the inner wall of the sleeve), or from about 0.5 to about 8 mm, or from about 0.5 to about 6 mm, or from about 0.5 to about 5 mm, or from about 0.5 to about 4 mm, or from about 0.5 to about 3 mm, or from about 0.5 to about 2 mm, or from about 0.5 to about 1 mm.

In an alternative embodiment of the invention the insert is tapered, such that the spacing between the insert and the tubular membrane may be divergent along the length of the membrane. The spacing and the amount of divergence varied, depending upon the gradient of the tapered insert, the laminar conditions/flow velocities desired, size distribution, etc. It will be understood by the person skilled in the art that depending upon the direction of taper, the spacing between the insert and the tubular membrane may be divergent or convergent along the length of the membrane. The use of a tapered insert may be advantageous in that a suitable taper may allow the laminar flow to be held constant for a particular formulation and set of flow conditions. Thus, the tapered insert may be used to control variation in drop size resulting from changes in fluid properties, such as viscosity, as the material concentration in the solvent increases through its path along the length of the membrane.

In an alternative embodiment of the invention the crossflow apparatus may comprise more than one tubular membrane located inside the outer tubular sleeve, i.e. a plurality of tubular membranes. When a plurality of tubular membranes is provided, each membrane may optionally have an insert, as herein described, located inside it. A plurality of membranes may be grouped as a cluster of membranes positioned alongside each other. Desirably the membranes are not in direct contact with each other. It will be understood that the number of membranes may vary depending upon, inter alia, the nature of the droplets to be produced. Thus, by way of example only, when a plurality of tubular membranes is present, the number of membranes may be from 2 to 100.

The inclined second inlet provided in the outer tubular sleeve will generally comprise a branch of the tubular sleeve and may be perpendicular to the longitudinal axis of the tubular sleeve. The position of the branch or second inlet may be varied and may depend upon the plane of the membrane. In one embodiment the position of the branch or second inlet will be substantially equidistant from the inlet and the outlet, although it will be understood by the person skilled in the art that the location of this second inlet may be varied. It is also within the scope of the present invention for more than one branch inlet to be provided. For example the use of a dual branch may suitably allow for bleeding the second phase during priming, or flushing for cleaning, or drainage/venting for sterilisation.

The inlet and outlet ends of the outer sleeve will generally be provided with a seal assembly. Although the seal assemblies at the inlet and outlet ends of the outer sleeve may be the same or different, preferably each of the seal assemblies is the same. Normal O-ring seals involve the O-ring being compressed between the two faces on which the seal is required—in a variety of geometries. Commercially available O-ring seals are provided with different groove options with standard dimensions. Each seal assembly will comprise a tubular ferrule provided with a flange at each end. A first flange, located at the end adjacent to the outer sleeve (when coupled) may be provided with a circumferential internal recess which acts as a seat for an O-ring seal. When the O-ring seal is in place, the O-ring seal is adapted to be located around the end of the insert (when present) and within a recess in the outer sleeve to seal against leakage of fluid from within any of the elements of the crossflow apparatus. However, the O-ring seal used in the present invention is designed to allow a loose fit as the membrane slides through the O-rings. This arrangement is advantageous in that it avoids two potential problems while installing the membrane tube:
(1) the potential for crushing the thin membrane tube during installation; and
(2) the potential for the thin membrane tube to cut off the curved surface of the O-ring.

With the O-ring seal used in the present invention, when the end ferrules are clamped onto the outer sleeve they squeeze the sides of the O-rings causing them to deform and press onto the outer surface of the tubular membrane and the inner surface of the sleeve, to form a seal. This requires careful dimensioning and tolerances.

However, it will be understood by the person skilled in the art that other means of making seal may suitably be used, for example, use of a screwed fitting tightened to a particular torque which would avoid the need for close tolerances; or clamping parts to a particular force followed by welding (which may be particularly suitable when using a plastic crossflow apparatus).

The internal diameter of the tubular membrane may be varied. In particular, the internal diameter of the tubular membrane may vary depending upon whether or not an insert is present. Generally, the internal diameter of the tubular membrane will be fairly small. In the absence of an insert the internal diameter of the tubular membrane may be from about 1 mm to about 10 mm, or from about 2 mm to about 8 mm, or from about 4 mm to about 6 mm. When the tubular membrane is intended for use with an insert, the internal diameter of the tubular membrane may be from about 5 mm to about 50 mm, or from about 10 mm to about 50 mm, or from about 20 mm to about 40 mm, or from about 25 mm to about 35 mm. Higher internal diameter of the tubular membrane may only be capable of being subjected to lower injection pressure. The upper limit of the internal diameter of the tubular membrane may depend upon, inter alia, the thickness of the membrane tube, since the cylinder needs to be able to cope with the external injection pressure, and whether it's possible to drill consistent holes through that thickness. The chamber inside the cylindrical membrane usually contains the second liquid phase.

In contrast to membrane emulsification using oscillating membranes, in the present invention the membrane, the sleeve and the insert are generally stationary.)

As described herein in prior art membranes, such as those described in WO2012/094595 comprise pores in the membrane that are conical or concave in shape. One example is that the pores in the membrane can be laser drilled. Laser drilled membrane pores or through holes will be substantially more uniform in pore diameter, pore shape and pore depth. The profile of the pores may be important, for example, a sharp, well defined edge around the exit of the pore is preferable. It may be desirable to avoid a convoluted path (such as results from sintered membranes) in order to minimise blockage, reduce feed pressures (cf. mechanical strength), and keep an even flowrate from each pore. However, as discussed herein, it is within the scope of the present invention to use pores in which the internal bore is non-circular (for example rectangular slots) or convoluted (for example tapered or stepped diameter to minimise pressure drop).

In the membrane the pores may be uniformly spaced or may have a variable pitch. Alternatively, the membrane pores may have a uniform pitch within a row or circumference, but a different pitch in another direction.

The pores in the membrane may vary. By way of example only, the pores in the membrane may have a pore diameter of from about 1 μm to about 100 μm, or about 10 μm to about 100 μm, or about 20 μm to about 100 μm, or about 30 μm to about 100 μm, or about 40 μm to about 100 μm, or about 50 μm to about 100 μm, or about 60 μm to about 100 μm, or about 70 μm to about 100 μm, or about 80 μm to about 100 μm, or about 90 μm to about 100 μm. In a further embodiment of the invention the pores in the membrane may have a pore diameter of from about 1 μm to about 40 μm, e.g. about 3 μm, or from about 5 μm to about 20 μm, or from about 5 μm to about 15 μm.

In the membrane the shape of the pores may be substantially tubular. However, it is within the scope of the present invention to provide a membrane with uniformly tapered pores. Such uniformly tapered pores may be advantageous in that their use may reduce the pressure drop across the membrane and potentially increase throughput/flux. It is also within the scope of the present invention to provide a membrane in which the diameter is essentially constant, but the internal bore is non-circular (for example rectangular slots) or convoluted (for example tapered or stepped diameter to minimise pressure drop), providing pores with a high aspect ratio.

The interpore distance or pitch may vary depending upon, inter alia, the pore size; and may be from about 1 μm to about 5,000 μm, or from about 1 μm to about 1,000 μm, or from about 2 μm to about 800 μm, or from about 5 μm to about 600 μm, or from about 10 μm to about 500 μm, or from about 20 μm to about 400 μm, or from about 30 μm to about 300 μm, or from about 40 μm to about 200 μm, or from about 50 μm to about 100 μm, e.g. about 75 μm.

The surface porosity of the membrane may depend upon the pore size and may be from about 0.001% to about 20% of the surface area of the membrane; or from about 0.01% to about 20%, or from about 0.1% to about 20%, or from about 1% to about 20%, or from about 2% to about 20%, or from about 3% to about 20%, or from about 4% to about 20%, or from about 5% to about 20, or from about 5% to about 10%.

The arrangement of the pores may vary depending upon, inter alia, pore size, throughput, etc. Generally, the pores may be in a patterned arrangement, which may be a square, triangular, linear, circular, rectangular or other arrangement. In one embodiment the pores are in a square arrangement.

It will be understood that the reactive mixing apparatus of the invention; and in particular the membrane, may comprise known materials, such as glass; ceramic;

metal, e.g. stainless steel or nickel; polymer/plastic, such as a fluoropolymer; or silicon. The use of metals, such as stainless steel or nickel, or polymer/plastic, such as a fluoropolymer is advantageous in that, inter alia, the apparatus and/or membranes may be subjected to sterilisation, using conventional sterilisation techniques known in the art, including gamma irradiation where appropriate. The use of polymer/plastic material, such as a fluoropolymer, is advantageous in that, inter alia, the apparatus and/or membrane may be manufactured using injection moulding techniques known in the art.

Thus, according a further aspect of the invention there is provided a reactive crystallisation apparatus for reactively dispersing a first phase in a second phase, comprising:
a membrane defining a plurality of apertures connecting a first liquid phase, wherein said first liquid phase comprises a solution of a first material, on a first side of the membrane; to a second liquid phase, wherein said second liquid phase comprises a solution of a second material, on a second different side of the membrane;
the apparatus being adapted to generate a reactive mixture through egression of the first liquid phase into the second liquid phase via the plurality of apertures;
the apparatus also comprising a reaction chamber arranged to receive the first and second liquid phases and/or the reactive mixture from the membrane.

As described herein an insert may be included in the membrane to facilitate even flow distribution. However, it is within the scope of the crossflow apparatus of the present invention for the insert to be absent. When an insert is present, the furcation plate may be adapted to split the flow of second phase or the first phase into a number of branches. Whether the furcation plate splits the second phase or the first phase will depend upon the direction of flow of the second phase, i.e. whether the second phase flows through the first inlet or the second inlet. Although the number of furcation plates may be varied, the number selected should be suitable lead to even flow distribution and (at the particle outlet end) not have excessive shear. Preferably, when the insert is present the furcation plate is a bi-furcation plate or a tri-furcation plate to provide a uniform second phase flow within the annular region between the insert and the membrane. Most preferably the furcation plate is a tri-furcation plate.

The number of orifices provided in the insert may vary depending upon the injection rate, etc. Generally the number of orifices may be from 2 to 6. Preferably the number of orifice is three.

The chamfered region on the insert is advantageous in that it enables the insert to be centred when it is located in position inside the membrane. The external circumference of the ends of the insert has a minimal tolerance with the internal diameter of the tubular membrane. This enables the insert to be accurately centred, thereby providing a consistent annulus leading to a consistent laminar flow. Generally, the chamfered region will comprise a shallow chamfer, which is advantageous in that it evens the flow distribution and allows the use of orifices in the insert with larger cross-sectional area than could be achieved if the flow simply entered through orifices parallel to the axis of the insert. This keeps the fluid velocity down and therefore minimises unwanted pressure losses, and shear on the outlet. The distance between the start of the orifices and the start of the porous region on the tubular membrane allows an even velocity distribution to be established. The radial dimension of the insert is selected to provide an annular depth to provide a certain laminar flow for the flowrates chosen. The axial dimension is designed to generally give a combined orifice area which is greater than both the annular area and the inlet/exit tube area.

The use of membrane emulsification techniques in the reactive preparation of solid materials as herein described may comprise the use of turbulent flow or the use of laminar flow, e.g. by stirring or liquid flow. In a particular aspect of the invention the reactive membrane emulsification technique comprises the use of laminar flow, i.e. whilst generally avoiding or minimising any turbulent flow.

The use of reactive membrane emulsification techniques in the preparation of solid materials as herein described may include the use of one or more pump systems. It will be understood that any conventionally known pumping system for use with reactive membrane emulsification may suitably be used. However, in a particular aspect of the invention the pump system may comprise a gear pump or a peristatic pump; and combinations thereof.

The method of the invention can be used to precisely control the distribution of chemical conditions and mechanical forces so that they are substantially constant on a length scale. Hence, resultant solid material particles are more uniform in size, with narrow size distribution.

The method of the invention may comprise a batch process or a continuous process. Desirably, the method of the invention may comprise a continuous process.

The reactive membrane emulsification apparatus may comprise a laboratory dispersion cell (LDC), which uses a precision engineered circular membrane, with a stirrer being used to generate the shear required for droplet formation; or a crossflow apparatus (AXF). When the AXF is used in a continuous flow method, it is generally referred to as Continuous Crossflow (CXF).

The solid particle size distribution may be measured by a variety of techniques. An exemplary technique is to measure the solid particle size distribution by laser diffraction, e.g. using a Malvern Mastersizer 2000 (Worcestershire, UK). The relative volume, Vi, of the particles in different size classes i, whose mean diameter di range from 0.01 to 3500 μm, may be used to calculate the volume-weighted mean diameter, d[4,3]:

$$d \vert 4 , 3 \vert = \frac{\sum {V}_{i} {d}_{i}}{\sum {V}_{i}}$$

The size uniformity of the solid particle was estimated using span of a particle size distribution:

$$span = \frac{[ d ( v , 0.9 ) - d ( v , 0.1 ) ]}{d ( v , 0.5 )}$$

where d (v, 0.1), d (v, 0.5), and d (v, 0.9) are the particle diameters at 10 vol %, 50 vol %, and 90 vol % of the cumulative distribution.

In one aspect of the invention the crossflow reactive mixing/emulsification apparatus includes an insert as herein described and the first inlet is a second phase first inlet and the second inlet is a first phase inlet; such that the first phase travels from outside the tubular membrane to inside.

In another aspect of the invention the crossflow reactive mixing/emulsification apparatus does not include an insert and the first inlet is a first phase first inlet and the second inlet is a second phase inlet; such that the first phase travels from inside the tubular membrane to outside.

Solid material particles prepared by the method of the invention are useful as components in pharmaceutical compositions. These compositions will typically include a pharmaceutically acceptable carrier in addition to the pharmaceutically active solid particles.

According to a further aspect of the invention there is provided the use of a membrane emulsification apparatus as a reactive mixing apparatus.

In particular, there is provided the use of a crossflow membrane emulsification apparatus as a reactive mixing apparatus

In the use according to this aspect of the invention the membrane emulsification apparatus is a reactive cross-flow emulsification apparatus.

There is further provided the use of a reactive cross-flow emulsification apparatus as a reactive mixing apparatus; said cross-flow emulsification apparatus comprising:
an outer tubular sleeve provided with a first inlet at a first end; an emulsion outlet; and a second inlet, distal from and inclined relative to the first inlet;
a tubular membrane provided with a plurality of pores and adapted to be positioned inside the tubular sleeve; and
optionally an insert adapted to be located inside the tubular membrane, said insert comprising an inlet end and an outlet end, each of the inlet end and an outlet end being provided with chamfered region; the chamfered region is provided with a plurality of orifices and a furcation plate.

Therefore, according to a further aspect of the present invention there is provided a material in solid particle form prepared by the method herein described. The material in solid particle form according to this aspect of the invention may be in crystalline form or amorphous form, or a combination thereof. In one aspect of the invention the material in solid particle form comprises solid crystalline particles. In another aspect of the invention the material in solid particle form comprises solid amorphous particles.

According to this aspect of the invention the material in solid particle form, e.g. crystalline or amorphous, may comprise an active agent.

By way of example only, active agents which comprise the solid particles of the present invention include, but shall not be limited to, biologically active agents, such as pharmaceutically active agents, pesticides and the like. Biologically active agents may also include, for example, a plant growth regulant. Alternatively, the active agent may be non-biologically active, such as, a plant nutritive substance, a food flavouring, a fragrance, and the like.

Pharmaceutically active agents refer to naturally occurring, synthetic, or semi-synthetic materials (e.g., compounds, fermentates, extracts, cellular structures) capable of eliciting, directly or indirectly, one or more physical, chemical, and/or biological effects, in vitro and/or in vivo. Such active agents may be capable of preventing, alleviating, treating, and/or curing abnormal and/or pathological conditions of a living body, such as by destroying a parasitic organism, or by limiting the effect of a disease or abnormality by materially altering the physiology of the host or parasite. Such active agents may be capable of maintaining, increasing, decreasing, limiting, or destroying a physiologic body function. Active agents may be capable of diagnosing a physiological condition or state by an in vitro and/or in vivo test. The active agent may be capable of controlling or protecting an environment or living body by attracting, disabling, inhibiting, killing, modifying, repelling and/or retarding an animal or microorganism. Active agents may be capable of otherwise treating (such as deodorising, protecting, adorning, grooming) a body. Depending upon the effect and/or its application, the active agent may further be referred to as a bioactive agent, a pharmaceutical agent (such as a prophylactic agent, or a therapeutic agent), a diagnostic agent, a nutritional supplement, and/or a cosmetic agent, and includes, without limitation, prodrugs, affinity molecules, synthetic organic molecules, proteinaceous compounds, peptides, vitamins, steroids, steroid analogues, nucleic acids, carbohydrates, precursors thereof and derivatives thereof. Active agents may be ionic, non-ionic, neutral, positively charged, negatively charged, or zwitterionic, and may be used singly or in combination of two or more thereof. Active agents may be water insoluble or water soluble.

A wide variety of pharmaceutically active agents may be utilised in the present invention. Thus, the pharmaceutically active agent may comprise one or more of a polynucleotide, a peptide, a protein, a small organic active agent, a small inorganic active agent and mixtures thereof.

In a particular aspect of the present invention the solid particles produced comprise a pharmaceutically active compound. It will be understood by the person skilled in the art that any suitably poorly soluble pharmaceutically active compound may be used in the method of the invention. Such pharmaceutically active compounds may include, but shall not be limited to, antifungal agents, such as, itraconazole fluoconazole, terconazole, ketoconazole and saperconazole; anti-infective agents, such as griseofulvin and griseoverdin; antibiotics, such as, amoxicillin, azithromycin, cephalexin, cefixime, cefoperazone, ceftriaxone, ciprofloxacin, clarithromycin, clavulanic acid, clindamycin, doxycycline, erythromycin, gentamycin, levofloxacin, meropenem, metronidazole, neomycin, norfloxacin, ofloxacin, ornidazole, oxytctracycline, piperacillin, rifampicin, streptomycin, sulbactam, sulfadiazine, tazobactam, tetracycline and tinidazole; anti malaria drugs, such as, atovaquone and artesunate; protein kinase inhibitors, such as, afatinib, axitinib, bosutinib, cetuximab, crizotinib, dasatinib, erlotinib, fostamatinib, gefitinib, ibrutinib, imatinib, zemurasenib, lapatinib, lenvatinib, mubritinib and nilotinib; immune system modulators, such as, cyclosporine; cardiovascular drugs, such as, digoxin and spironolactone; sterols or steroids, such as, betamethasone; ACE inhibitors, such as, captopril, enalapril, ramipril, quinapril, perindopril, lisinopril, and fosinopril; adenohypophyseal hormones; adrenergic antagonists, such as, phentolamine, phenoxybenzamine, tamsulosin, propranolol, atenolol, metoprolol, timolol and acebutolol; adrenocortical steroids; inhibitors of the biosynthesis of adrenocortical steroids; alpha-adrenergic agonists, such as methoxamine, phenylephrine, methyldopa, norepinephrine; alpha-adrenergic antagonists, such as, phentolamine and phenoxybenzamine; analgesics, such as, aspirin and paracetamol; antipyretics and anti-inflammatory agents, such as, diclofenac, ibuprofen, naproxen and ketoprofen; androgens, local anaesthetics, such as, lidocaine; antiaddictive agents; antiandrogens; antiarrhythmic agents, such as, verapamil and diltiazem; antiasthmatic agents, such as, beclomethasone, budesonide, fluticasone, reproterol, salbutamol and salmeterol; anticholinergic agents, such as, ipratropium and oxybutynin; anticholinesterase agents, such as, donepezil; anticoagulants, such as, dabigatran, rivaroxaban, apixaban, edoxaban and betrixaban; antidiabetic agents, such as, metformin; antidiarrheal agents; antidiuretics; antiemetic and prokinetic agents; antiepileptic agents, such as carbamazepine, gabapentin oxcarbazepine; antiestrogens; antifungal agents; antihypertensive agents, such as, losartan, olmesartan, telmisartan and valsartan; antimicrobial agents; antimigraine agents, such as, zolmitriptan; antimuscarinic agents; antineoplastic agents; antiparasitic agents; antiparkinsons agents, such as, carbidopa and levodopa; antiplatelet agents; antiprogestins; antithyroid agents; antitussives; antiviral agents; antidepressants; azaspirodecanediones; barbiturates; benzodiazepines; benzothiadiazides; beta-adrenergic agonists; beta-adrenergic antagonists; selective adrenergic antagonists; selective agonists; bile salts; butyrophenones; calcium channel blockers; catecholamines and sympathomimetic drugs; cholinergic agonists; cholinesterase reactivators; cognitive enhancers, such as, piracetam; dermatological agents; diphenylbutylpiperidines; diuretics; ergot alkaloids; oestrogens; ganglionic blocking agents; ganglionic stimulating agents; glucocorticoid steroids, such as, dexamethasone and prednisolone; agents for control of gastric acidity and treatment of peptic ulcers; haematopoietic agents; histamines; antihistamine; HMG-COA reductase inhibitors, e.g. statins, such as, atorvastatin, fluvastatin, lovastatin, pitavastatin, pravastatin, rosuvastatin and simvastatin; 5-hydroxytryptamine antagonists; hypnotics and sedatives; immunosuppressive agents; laxatives; methylxanthines; monoamine oxidase inhibitors; neuromuscular blocking agents; nutrients or dietary supplements, such as, vitamin B1, vitamin B6 and retinol; organic nitrates; opioid analgesics and antagonists; pancreatic enzymes; phenothiazines; progestins; prostaglandins; agents for the treatment of psychiatric disorders; retinoids; sodium channel blockers; thrombolytic agents; thyroid agents; tricyclic antidepressants; tyrosine kinase inhibitors, such as, axitinib, crizotinib, dasatinib, erlotinib, gefitinib, imatinib, lapatinib, nilotinib, pazopanib, regorafenib, ruxolitinib, sorafenib, sunitinib, vandetanib and vemurafenib; drugs from the group comprising danazol, acyclovir, dapsone, indinavir, lopinavir, nifedipine, nitrofurantoin, phentytoin, ritonavir, saquinavir, sulfamethoxazole, valproic acid, trimethoprin, acetazolamide, azathioprine, iopanoic acid, nalidixic acid, nevirapine, praziquantel, rifampicin, albendazole, amitriptyline, artemether, lumefantrine, chloropromazine, clofazimine, efavirenz, iopinavir, folic acid, glibenclamide, haloperidol, ivermectin, mebendazole, niclosamide, pyrantel, pyrimethamine, sulfadiazine, sulfasalazine, triclabendazole, and cinnarizine; and combinations thereof. Such pharmaceutically active compounds may be in free form or salt form.

Particles obtained by the method of the present invention may be formulated into a pharmaceutical composition. Examples of pharmaceutical forms for administration of solid particles prepared using the methods herein described may include solid dosage forms, such as, tablets, capsules, granules, pellets or powders. The compositions obtained may have an enhanced performance including, but not exclusively, supersaturation, improved dissolution rate, improved bioavailability, improved or controlled release, and the like.

Particles obtained by the method of the present invention may be formulated into a pharmaceutical formulation in the form of excipients. An excipient is a substance formulated alongside the pharmaceutical active ingredient, included for the purpose of long-term stabilization, bulking up solid formulations or therapeutic enhancement on the active ingredient in the final dosage form. The excipient can aid in other areas such as facilitating drug absorption, reducing viscosity, or enhancing solubility.

The present invention will now be described by way of example only with reference to the accompanying figures in which:

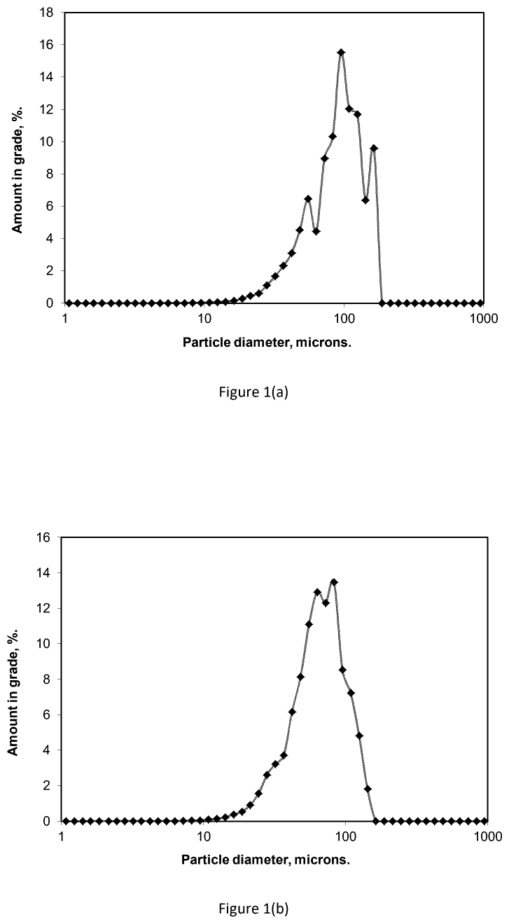

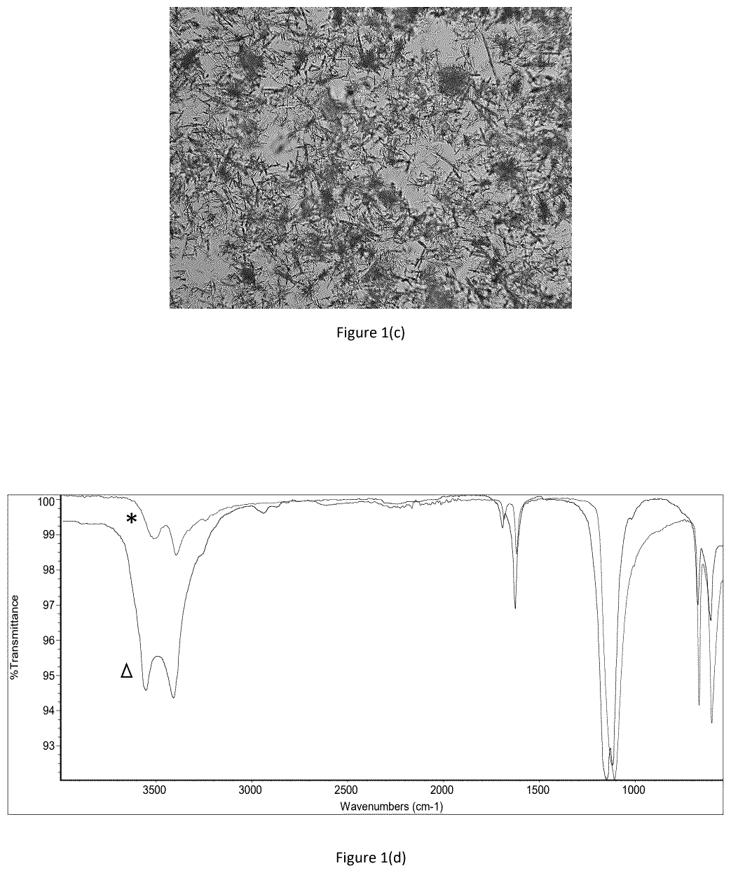

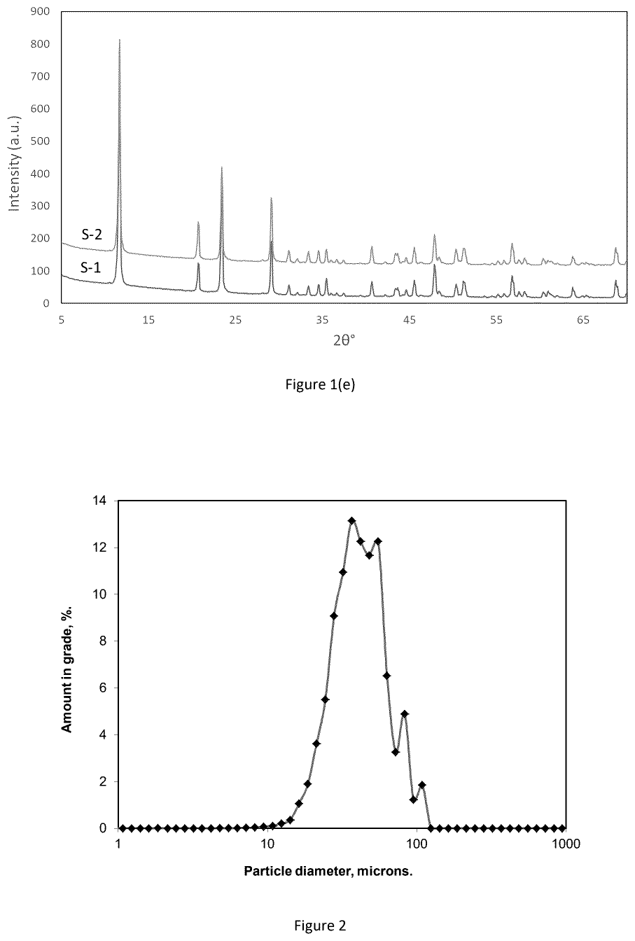

FIG. 1(a) illustrates the particle diameters for CaSO₄·2H₂O via batch crystallisation;

FIG. 1(b) illustrates the particle diameters for CaSO₄·2H₂O via reactive crystallisation using AXF mini;

FIG. 1(c) illustrates microscopic images for reactively crystallised CaSO₄·2H₂O (×100 magnification);

FIG. 1(d) illustrates FTIR spectra for reactive crystallised CaSO₄·2H₂O (*) with vibrational peaks in the spectrum matching those of the reference CaSO₄·2H₂O (A) obtained from NIST;

FIG. 1(e) illustrates X-ray diffraction patterns of S-1) batch reactive crystallisation of CaSO₄·2H₂O, S-2) AXF-mini reactive crystallisation of CaSO₄·2H₂O;

FIG. 2 illustrates particle diameters for CaHPO₄·2H₂O crystallisation using batch reactive crystallisation;

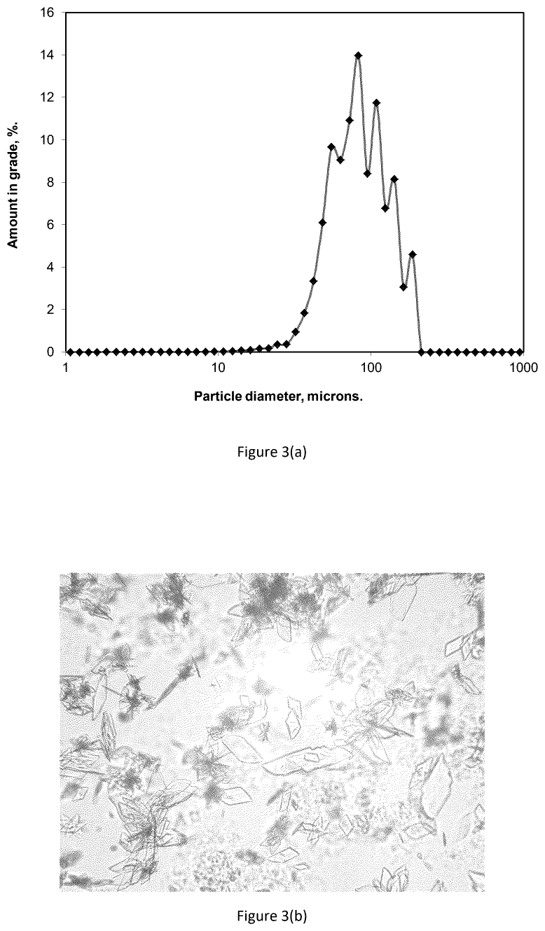

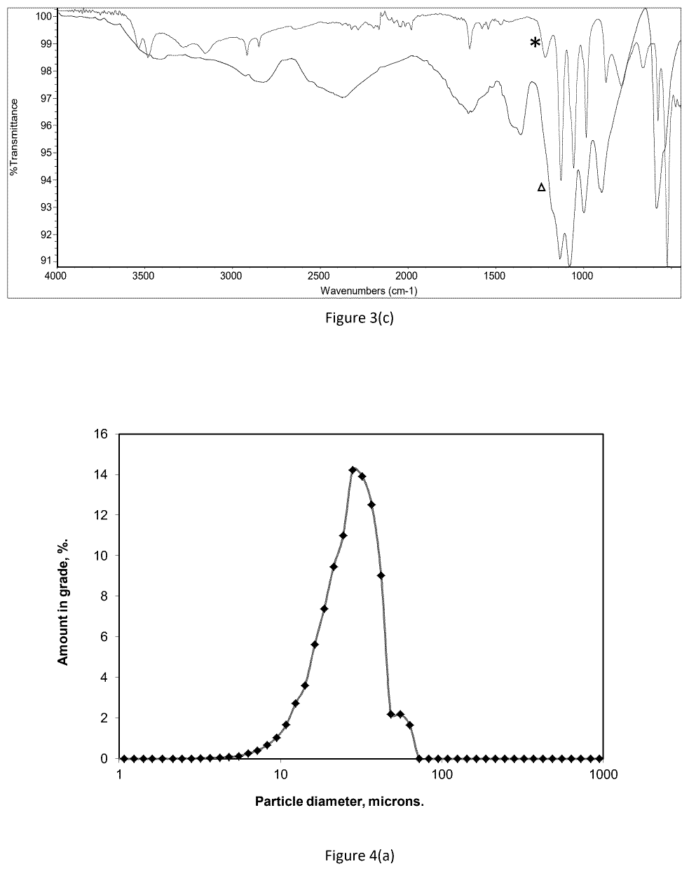

FIG. 3(a) illustrates particle diameters for CaHPO₄·2H₂O crystallisation using AXF-mini stoichiometric reactive crystallisation with CP:DP at 5:1 ml/min;

FIG. 3(b) illustrates microscopic images for reactive crystallised CaHPO₄·2H₂O (×100 magnification);

FIG. 3(c) illustrates FTIR spectra for reactive crystallised CaHPO₄·2H₂O (*) with vibrational peaks in the spectrum matching those of the reference CaHPO₄·2H₂O (Δ) obtained from NIST;

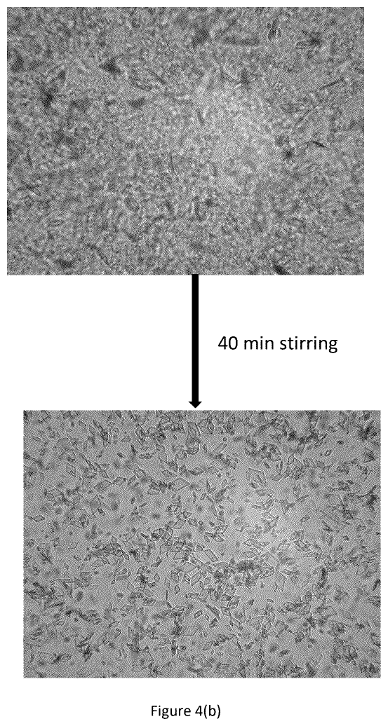

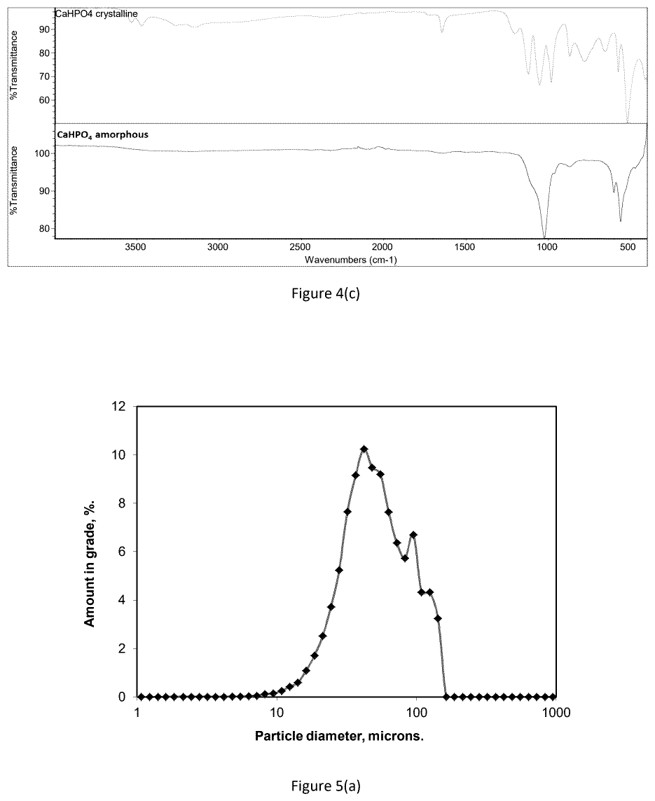

FIG. 4(a) illustrates particle diameters for CaHPO₄·2H₂O crystallisation using AXF-mini via non-stoichiometric reactive crystallisation with CP:DP at 10:0.5 ml/min;

FIG. 4(b) illustrates microscopic images of CaHPO₄·2H₂O crystals from reactive crystallisation using AXF-mini via non-stoichiometric reaction with CP:DP 10:0.5 ml/min (×100 magnification). Initial images show amorphous CaHPO₄·2H₂O crystals with stirring causing crystalline CaHPO₄·2H₂O crystals to form;

FIG. 4(c) illustrates FTIR spectra of amorphous and crystalline CaHPO₄·2H₂O from reactive crystallisation using AXF mini (non-Stoichiometric reaction concentrations);

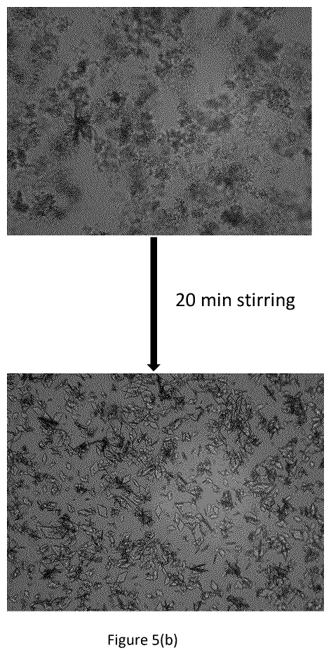

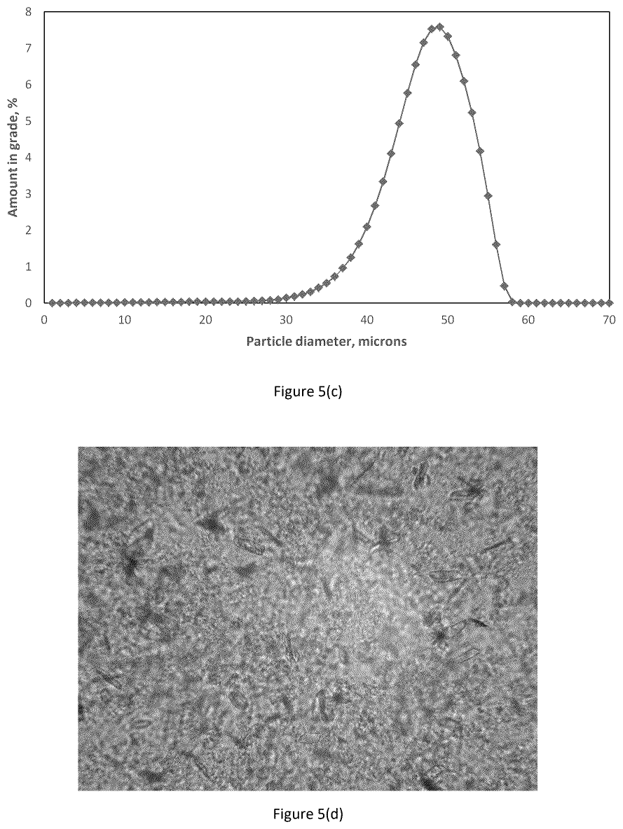

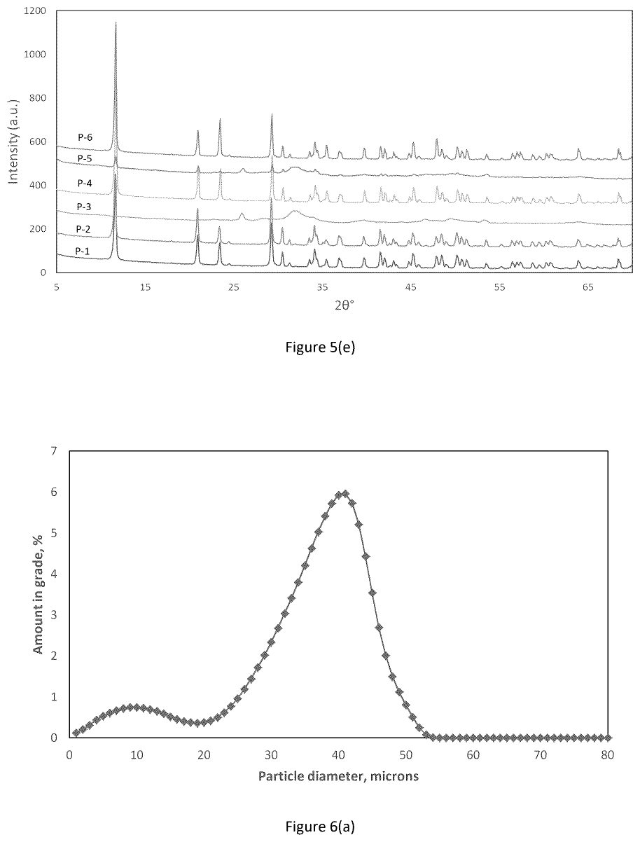

FIG. 5(a) illustrates particle diameters for CaHPO₄·2H₂O crystallisation using AXF-mini via stoichiometric reactive crystallisation with CP:DP at 10:0.5 ml/min;

FIG. 5(b) illustrates microscopic images of CaHPO₄·2H₂O crystals from reactive crystallisation using AXF-mini via stoichiometric reaction with CP:DP 10:0.5 ml/min (×100 magnification). Initial images show amorphous CaHPO₄·2H₂O crystals with stirring causing crystalline CaHPO₄·2H₂O crystals to form;

FIG. 5(c) illustrates particle diameters for CaHPO₄·2H₂O crystallisation using AXF-mini via stoichiometric reactive crystallisation with CP:DP at 10:0.5 ml/min;

FIG. 5(d) illustrates microscopic images for amorphous CaHPO₄·2H₂O from stoichiometric reactive crystallisation using AXF mini (×100 magnification);

FIG. 5(e) illustrates X-ray diffraction patterns of P-1) batch reactive crystallisation of CaHPO₄·2H₂O, P-2) LDC-1 reactive crystallisation of CaHPO₄·2H₂O, P-3) AXF-mini reactive crystallisation of amorphous non-stoichiometric Ca₅(PO₄)₃OH, P-4) AXF-mini reactive crystallisation of crystalline non-stoichiometric CaHPO₄·2H₂O, P-5) AXF-mini reactive crystallisation of amorphous stoichiometric CaHPO₄·2H₂O, P-6) AXF-mini reactive crystallisation of crystalline stoichiometric CaHPO₄·2H₂O;

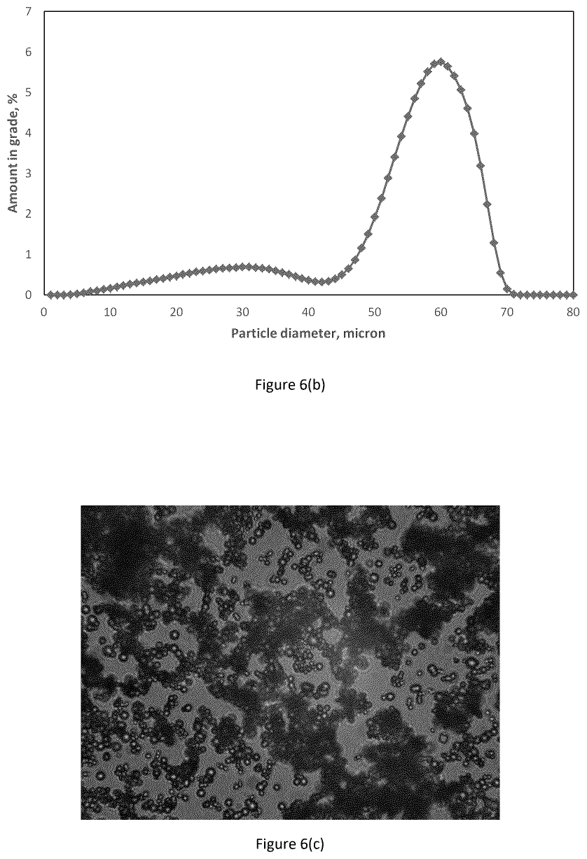

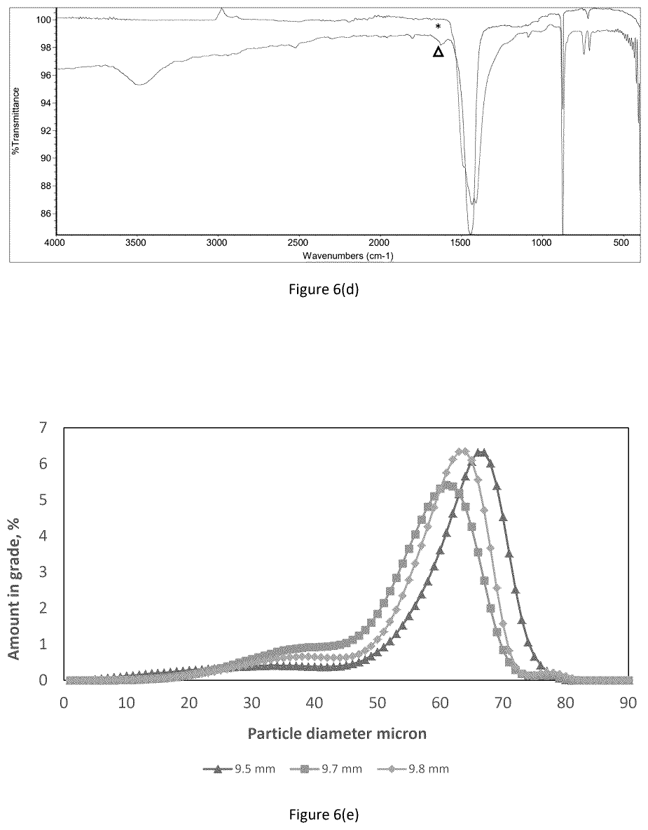

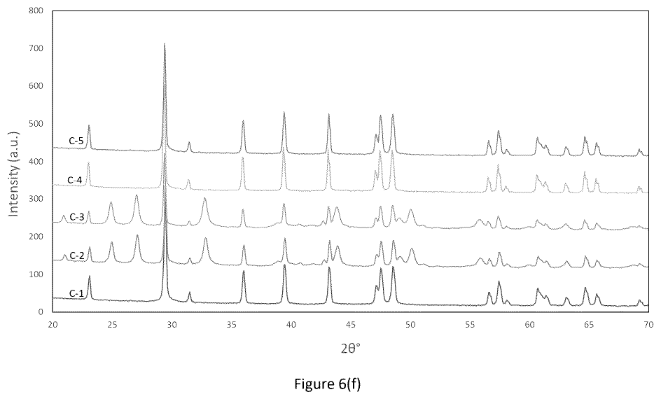

FIG. 6(a) illustrates particle diameters for CaCO₃ from batch reactive crystallisation;

FIG. 6(b) illustrates particle diameters for CaO₃ using AXF-mini reactive crystallisation;

FIG. 6(c) illustrates microscopic images for reactively crystallised CaCO₃ (×400 magnification);

FIG. 6(d) illustrates FTIR spectra for reactive crystallised CaCO₃ (*) with vibrational peaks in the spectrum matching those of the reference CaCO₃ (Δ) obtained from NIST;

FIG. 6(e) illustrates particle diameters for CaCO₃ from reactive crystallisation using AXF-1 with flow rates of 250:50 ml/min using 10×200 μm membrane and inserts of 9.5, 9.7 and 9.8 mm; and

FIG. 6(f) illustrates X-ray diffraction patterns of C-1) batch reactive crystallisation of CaO₃ calcite and vaterite mixture, C-2) AXF-mini reactive crystallisation of CaO₃ calcite and vaterite, C-3) AXF-1 with 9.5 mm insert reactive crystallisation of CaO₃ calcite and vaterite, C-4) AXF-1 with 9.7 mm insert reactive crystallisation of CaO₃ calcite, C-5) AXF-1 with 9.8 mm insert reactive crystallisation of CaO₃ calcite.

As a case study the formation of CaSO₄·2H₂O, CaHPO₄·2H₂O and CaCO₃ has been carried out.

CaSO₄·2H₂O, CaHPO₄·2H₂O and CaCO₃ are all readily used in the pharmaceutical industry as excipients (inactive ingredient). Good size and CSD control makes the blending and mixing with Active Pharmaceutical Ingredients (APIs) better as well as the general particle handling process.

Other applications of these materials include, but shall not be limited to:
CaSO₄·2H₂O—construction materials, fertilizers, dentistry fillers;
CaHPO₄·2H₂O—treatment of wastewater and polluted soil, source of calcium and phosphorus, high-end nutrients; and
CaCO₃—fillers in adhesives and sealants, plastics, paper manufacturing, paints and inks, catalysts and gas filters.

Inorganic reactive crystallisation studies were conducted with the following reactions:
CaCl₂+Na₂SO₄→CaSO₄·2H₂O+2NaCl
CaCl₂)+NaH₂PO₄·2H₂O→CaHPO₄·2H₂O+NaCl+HCl+2H₂O
CaCl₂+Na₂CO₃→CaCO₃+2NaCl

All reactions were carried out in aqueous conditions and based on solubility. For example, CaCl₂, Na₂SO₄, NaH₂PO₄·2H₂O, Na₂CO₃, NaCl and HCl are all soluble in water. CaSO₄·2H₂O, CaHPO₄·2H₂O and CaCO₃ are all poorly soluble in water.

### Example 1

CaSO₄·2H₂O Crystallisation

Reactive crystallisation was conducted via a standard batch process then in a mini-crossflow membrane emulsification apparatus (AXF mini). This had the continuous phase (CP) and dispersed phase (DP) contain the reagents that would undergo reactive crystallisation towards CaSO₄·2H₂O. This was compared to a batch reactive crystallisation process to observe the benefits of the crossflow membrane methodology.

### 1.1 Batch Run

14 V stirrer, 10 ml/min rate
DP CaCl₂)/DI water 0.100 g/ml, 10 ml added
CP Na₂SO₄/Di water 0.0256 g/ml, 50 ml total
Flux 1842.2 ml/min/cm2, Shear 24.577 Pa

### 1.2 AXF-Mini Run

5×100 μm membrane, 9.8 mm
Flow rates-CP 5 ml/min, DP 1 ml/min
Pore Velocity (m/s)=0.0270, Annulus Velocity (m/s)=0.0268
Momentum Flux Ratio=1.02, Re (annulus)=5.36

The results are provided in FIGS. 1(a) to 1(e).

FTIR spectroscopy and X-ray diffraction analysis show reactive crystallisation of CaSO₄·2H₂O has been successful using both batch and continuous AXF-mini runs. Particle size analysis shows the AXF-mini apparatus has reduced the size of the crystals.

### Example 2

CaHPO₄·2H₂O Crystallisation

Reactive crystallisation was conducted via a standard batch process than in a mini-crossflow membrane emulsification apparatus (AXF mini). This had the continuous phase (CP) and dispersed phase (DP) contain the reagents that would undergo reactive crystallisation towards CaHPO₄·2H₂O. This was compared to a batch reactive crystallisation process to observe the benefits of the crossflow membrane methodology.

### 2.1 Batch Process

DP CaCl₂)/DI water 0.100 g/ml, 10 ml added
CP NaH₂PO₄·2H₂O/Di water 0.0281 g/ml, 50 ml total
Flux 1842.2 ml/min/cm2, Shear 24.577 Pa

Note—CP solution was raised to pH=6.51 with 4M NaOH, this was due to CaHPO₄·2H₂O being unable to precipitate under acidic conditions 14 V stirring, 10 ml/min rate

The results are provided in FIG. 2.

### 2.2 AXF Mini Run

DP CaCl₂)/DI water 0.100 g/ml,
CP NaH₂PO₄·2H₂O/Di water 0.0281 g/ml,

10×200 μm membrane, 9.8 mm membrane

Note—CP solution was raised to pH=6.51 with 4M NaOH

Both stoichiometric and non-stoichiometric reactions were carried out

### Stoichiometric (5:1) Reaction

5 parts NaH₂PO₄·2H₂O solution reacting with 1 part CaCl₂) solution based on flow rate and concentration.

Pore Velocity (m/s)=0.0270, Annulus Velocity (m/s)=0.0268

Momentum Flux Ratio=1.02, Re (annulus)=5.36

### Non-Stoichiometric (10:0.5) Reaction

20 parts NaH₂PO₄·2H₂O solution reacting with 1 part CaCl₂) solution based on flow rate and concentration.

Pore Velocity (m/s)=0.0135, Annulus Velocity (m/s)=0.0536

Momentum Flux Ratio=0.0636, Re (annulus)=10.72

The results are provided in FIGS. 3(a) to 4(c).

### 2.3 AXF Mini Run

DP CaCl₂/DI water 0.200 g/ml, 0.5 ml/min
CP NaH₂PO₄·2H₂O/Di water 0.01425 g/ml, 10 ml/min

10×200 μm membrane, 9.8 mm membrane

Note—CP solution was raised to pH=6.51 with 4M NaOH

### Stoichiometric (5:1) Reactions

CP:DP flow rates 10:0.5 ml/min

5 parts NaH₂PO₄·2H₂O solution reacting with 1 part CaCl₂ solution based on flow rate and concentration.

Pore Velocity (m/s)=0.0135, Annulus Velocity (m/s)=0.0536

Momentum Flux Ratio=0.0636, Re (annulus)=10.72

The results are provided in FIGS. 5(a) to 5(e).

FTIR spectroscopy and X-ray diffraction analysis showed reactive crystallisation of CaHPO₄·2H₂O has been successful using both batch and continuous AXF-mini runs towards crystalline materials. The continuous AXF-mini setup initially produced amorphous crystals that under stirring would arrange into long-ranged ordered crystalline forms. The different crystalline forms were also confirmed using FTIR spectroscopy and X-ray diffraction analysis. XRPD analysis showed the amorphous non-stoichiometric material was Ca₅(PO₄)₃OH whilst the amorphous stoichiometric material was a mixture of CaHPO₄·2H₂O and Ca₅(PO₄)₃OH.

Particle size analysis shows the AXF-mini apparatus has reduced the size of the crystals and improved the CSD compared to batch. This improvement was possible either through changing the concentrations and flow rate for a stoichiometric reaction. Or through just changing the flow rate resulting in a non-stoichiometric reaction.

### Example 3

### CaCO₃ Crystallisation

Reactive crystallisation was conducted in a mini-crossflow membrane emulsification apparatus (AXF mini) and a crossflow membrane emulsification apparatus (AXF). This had the continuous phase (CP) and dispersed phase (DP) contain different reagents that would undergo reactive crystallisation towards CaCO₃. The scaled up of equipment and reactive crystallisation was shown to be successful with repeatable particle size results and levels of purity.

### 3.1 Batch Process

DP CaCl₂/DI water 0.100 g/ml, 10 ml added
CP NaCO₃/Di water 0.0212 g/ml, 50 ml total
Flux 1842.2 ml/min/cm2, Shear 24.577 Pa

The results are provided in FIG. 6(a).

### 3.2 AXF Mini Run

DP CaCl₂/DI water 0.111 g/ml,
CP Na₂CO₃/Di water 0.0212 g/ml,

10×200 μm membrane, 9.8 mm

Flow rates-CP 5 ml/min, DP 1 ml/min

Pore Velocity (m/s)=0.0270, Annulus Velocity (m/s)=0.0268

Momentum Flux Ratio=1.02, Re (annulus)=5.36

The results are provided in FIGS. 6(b) to 4(d).

### 3.3 AXF-1 Run

DP CaCl₂/DI water 0.111 g/ml,
CP Na₂CO₃/Di water 0.0212 g/ml,

10×200 μm membrane, 9.8, 9.7 and 9.5 mm inserts

Flow rates—CP=250 ml/min, DP=50 ml/min

### 9.5 mm Insert

Pore Velocity (m/s)=0.135, Annulus Velocity (m/s)=0.544

Momentum Flux Ratio=0.0616, Re (annulus)=272.06

### 9.7 mm Insert

Pore Velocity (m/s)=0.135, Annulus Velocity (m/s)=0.898

Momentum Flux Ratio=0.0226, Re (annulus)=269.30

### 9.8 mm Insert

Pore Velocity (m/s)=0.135, Annulus Velocity (m/s)=1.340

Momentum Flux Ratio=1.02, Re (annulus)=267.94

The results are provided in FIGS. 6(e) to 6(f).

FTIR spectroscopy and X-ray diffraction analysis showed reactive crystallisation of precipitated CaCO₃ has been successful through AXF-mini runs with a mixture of calcite and vaterite polymorphs. The scale up of the reactive crystallisation through the AXF-1 has been investigated and was shown to be successful with repeatable crystal sizes to the AXF-mini results. This was possible through investigating different insert sizes and the effect these have on the crystal sizes and overall reactive crystallisation process. It was found that inserts of 9.7 and 9.8 mm diameter would result in pure calcite formation, however, would eventual result in blockages of the CaCO₃ crystal flow. However, when an insert of 9.5 mm was used this was ran with no blockages observed but the XRPD result showed a mixture of vaterite and calcite.

1. A method of preparing solid particles of a material, said method comprising controlling provision of a first liquid phase, wherein said first liquid phase comprises a solution of a first material through a membrane, said membrane defining a plurality of pores; and controlling provision of a second liquid phase, wherein said second liquid phase comprises a solution of a second material; reacting the first a second materials to produce a third liquid phase comprising a solution of a third material; and supersaturating the third liquid phase to form solid particles of the third material.

2. A method of preparing solid particles of a material, said method comprising controlling provision of a first liquid phase, wherein said first liquid phase comprises a solution of a first material, in a first flow direction to a membrane, said membrane defining a plurality of pores; and
controlling provision of a second liquid phase, wherein said second liquid phase comprises a solution of a second material;
introduction of the second liquid phase being substantially perpendicular to the introduction of the first liquid phase;
reacting the first and second materials to produce a third liquid phase comprising a solution of a third material; and
supersaturating the third liquid phase to form solid particles of the third material.

3. A method according to claim 1 wherein the method comprises reactive preparation of solid particles of a material in crystalline form or amorphous form, or a combination thereof.

4. (canceled)

5. (canceled)

6. A method of reactive preparation of solid particles of a third material, said method comprising reacting a first liquid phase with a second liquid phase by dispersing the first liquid phase in the second liquid phase;
wherein said first liquid phase comprises a solution of a first material and said second liquid phase comprises a solution of a second material;
said method comprising controlling provision of the first liquid phase in a first flow direction to a membrane, said membrane defining a plurality of pores; and controlling provision of the second liquid phase to the membrane in a crossflow (AXF) to the first flow direction, via the plurality of pores, to reactively form a solution of a third material; and
optionally supersaturating the solution of the third material in order to produce particles of the third material.

7. A method according to claim 6 wherein the “crossflow” of the second liquid phase is at an angle of 90 degrees to the flow direction of the first liquid phase, plus or minus 45 degrees.

8. (canceled)

9. (canceled)

10. A method according to claim 1 wherein the method comprises preparing solid particles of more than one material, e.g. as co-crystals, comprising two or more components.

11. A method according to claim 1 wherein the prepared reactive solution (i.e. after reaction) includes one or more dissolved materials wherein the dissolved materials include one or more organic compounds, which may include, for example, pharmaceutically active compounds, bioactive agents, nutraceuticals, polymers and the like; metal-organic frameworks; or inorganic materials.

12.-14. (canceled)

15. A method of according to claim 1 wherein the method comprises the reactive preparation of solid amorphous particles of a material.

16. A method according to claim 1 wherein the dissolved prepared material (i.e. after reaction) comprises a material of low solubility.

17. A method according to claim 1 wherein the prepared reacted solution (i.e. after reaction) includes a dissolved material, wherein the material comprises one or more organic compounds, the one or more organic compounds comprising pharmaceutically active compounds or drugs, bioactive agents, nutraceuticals, polymers and the like.

18. (canceled)

19. A method according to claim 1817 wherein the solution comprises a pharmaceutically active compound of low bioavailability.

20. (canceled)

21. A method according to claim 1 wherein the prepared reacted solution (i.e. after reaction) include a dissolved material, wherein the material is an inorganic material.

22. (canceled)

23. (canceled)

24. A method according to claim 1 wherein the apparatus includes an insert.

25-35. (canceled)

36. A method according to claim 1 wherein the crossflow apparatus comprises a plurality of tubular membranes.

37. (canceled)

38. A method according to claim 14 wherein a plurality of membranes is grouped as a cluster of membranes positioned alongside each other.

39.-42. (canceled)

43. A method according to claim 1 wherein the membrane pores are laser drilled.

44.-47. (canceled)

48. A method according to claim 16 wherein the interpore distance is from about 1 μm to about 5,000 μm.

49. A method according to claim 16 wherein the surface porosity of the membrane may be from about 0.001% to about 20% of the surface area of the membrane.

50.-55. (canceled)

56. A method according to claim 81 wherein the furcation plate is a bi-furcation plate or a tri-furcation plate.

57.-67. (canceled)

68. A reactive crystallisation apparatus for reactively dispersing a first phase in a second phase, comprising:
a membrane defining a plurality of apertures connecting a first liquid phase, wherein said first liquid phase comprises a solution of a first material, on a first side of the membrane; to a second liquid phase, wherein said second liquid phase comprises a solution of a second material, on a second different side of the membrane;
the apparatus being adapted to generate a reactive mixture through egression of the first liquid phase into the second liquid phase via the plurality of apertures;
the apparatus also comprising a reaction chamber arranged to receive the first and second liquid phases and/or the reactive mixture from the membrane.

69.-73. (canceled)
